(19) United States
(12) Patent Application Publication
(10) Pub. No.: US 2016/0361061 A1
(43) Pub. Date: Dec. 15, 2016

(54) DEGRADABLE SURGICAL STAPLE

(71) Applicant: DONGGUAN EONTEC CO., LTD., Dongguan (CN)

(72) Inventor: Yangde LI, Dongguan (CN)

(21) Appl. No.: 15/246,575

(22) Filed: Aug. 25, 2016

Related U.S. Application Data
(63) Continuation-in-part of application No. PCT/CN2014/077955, filed on May 21, 2014.

(30) Foreign Application Priority Data
Feb. 26, 2014 (CN) 201410068125.8

(51) Int. Cl. A61B 17/064; A61B 17/115; A61B 17/11

(52) U.S. Cl. CPC A61B 17/0644; A61B 17/1114; A61B 17/115; A61B 2017/00004

(57) ABSTRACT

A degradable staple, including a base plate made from magnesium alloy. The surface of the base plate is provided with a plurality of opposite-distributed and staggered barbs. The base plate having the barbs on the surface can be rolled inward or outward to form a tube for stapling of gastrointestinal organs.

### CROSS-REFERENCE TO RELATED APPLICATIONS

This application is a continuation-in-part of International Patent Application No. PCT/CN2014/077955 with an international filing date of May 21, 2014, designating the United States, now pending, and further claims priority benefits to Chinese Patent Application No. 201410068125.8 filed Feb. 26, 2014. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, and Cambridge, MA 02142.

### BACKGROUND OF THE INVENTION

Field of the Invention

The invention relates to a degradable surgical staple made from magnesium alloy.

Description of the Related Art

As shown in FIG. 1, a conventional surgical staple for stapling tubular organs is tubular in shape and includes a plurality of barbs on the surface of the staple. The barbed surfaces are difficult to produce. In addition, the staple often has specific dimensions and size, and may not exactly correspond to the organs being stapled, such as gastrointestinal tubes. This reduces the stapling efficiency.

### SUMMARY OF THE INVENTION

In view of the above-described problems, it is one objective of the invention to provide a degradable surgical staple made from magnesium alloy which is convenient for use and applicable for the stapling of gastrointestinal organs.

The base plate of the staple is provided with a plurality of opposite-distributed and staggered barbs.

When used during surgery, a staple is cut according to the size of the gastrointestinal organs, and optionally, the base plate is rolled inward to form a tube, and the barbs protrude inward from the inner surface of the tube, then the gastrointestinal organ is sheathed in the tube, and hooked by the staggered barbs; or the base plate is rolled outward to form a tube, and the barbs protrude outward from the outer surface of the tube, then the gastrointestinal organ is sleeved on the tube, and hooked by the staggered barbs, thereby facilitating the stapling of gastrointestinal organ.

To achieve the above objective, in accordance with one embodiment of the invention, there is provided a degradable staple made from magnesium alloy for medical use, comprising a base plate made from magnesium alloy. The base plate comprises a surface on which a plurality of opposite-distributed and staggered barbs is disposed.

In a class of this embodiment, the base plate and surfaces of the barbs are coated with a drug layer; the drug layer is absorbable by human body.

In a class of this embodiment, the drug layer is a zinc ion layer. Optionally, the zinc ion layer is replaced by a coating made from biocompatible and corrosion-resistant material, or a coating made from anti-clotting, bactericidal, and bacteriostatic material.

In a class of this embodiment, the base plate having the barbs on the surface operates to roll inward or outward to form a tube for stapling of gastrointestinal organs.

Advantages of the degradable staple according to embodiments of the invention are summarized as follows:

The staple is applicable for the gastrointestinal stapling in human body. In operation, the staple is cut according to the size of the gastrointestinal organs. Optionally, the base plate is rolled inward to form a tube, and the barbs protrude inward from an inner surface of the tube, then two ends of the gastrointestinal organ are sheathed in the tube, slightly pulled, and thus hooked by the staggered barbs; or the base plate is rolled outward to form a tube, and the barbs protrude outward from an outer surface of the tube, then two ends of the gastrointestinal organ are sleeved on the tube, slightly pulled, and thus hooked by the staggered barbs, thereby facilitating the stapling of gastrointestinal organ. The base plate in a tube shape is degraded or absorbed by the human body as the wound gradually heals up.

The base plate and surfaces of the barbs are coated with a drug layer which can be absorbed by the body. The drug layer can be replaced by a coating made from biocompatible and corrosion-resistant material, or a coating made from anti-clotting, bactericidal, and bacteriostatic material. The drug layer and the coating bring no harm to the human body, instead, the drug layer and the coating with strong bactericidal capacity can protect the gastrointestinal organs from infections, thereby shortening the healing time of the wound.

### BRIEF DESCRIPTION OF THE DRAWINGS

The invention is described hereinbelow with reference to the accompanying drawings, in which:

FIG. 1 is a schematic diagram of a degradable staple made from magnesium alloy for medical use in the prior art;

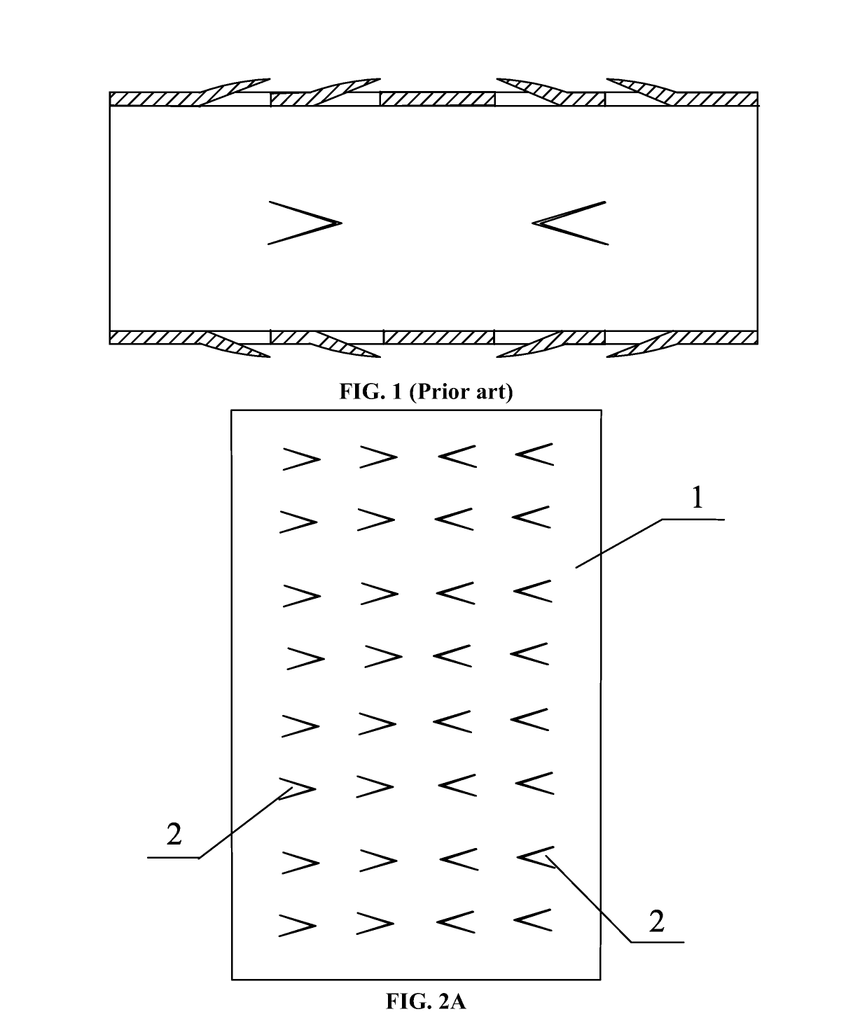

FIGS. 2A and 2B are schematic diagrams of a degradable surgical staple made from magnesium alloy; and

FIGS. 3-5 are diagrams showing stapling of gastrointestinal organs using a degradable surgical staple made from magnesium alloy.

In the drawings, the following reference numbers are used: 1. Base plate; 2. Barbs; 3. Drug layer; 4. Gastrointestinal organ; 5. Stapling nails.

### DETAILED DESCRIPTION OF THE EMBODIMENTS

For further illustrating the invention, experiments detailing a degradable surgical staple made from magnesium alloy are described below. It should be noted that the following examples are intended to describe and not to limit the invention.

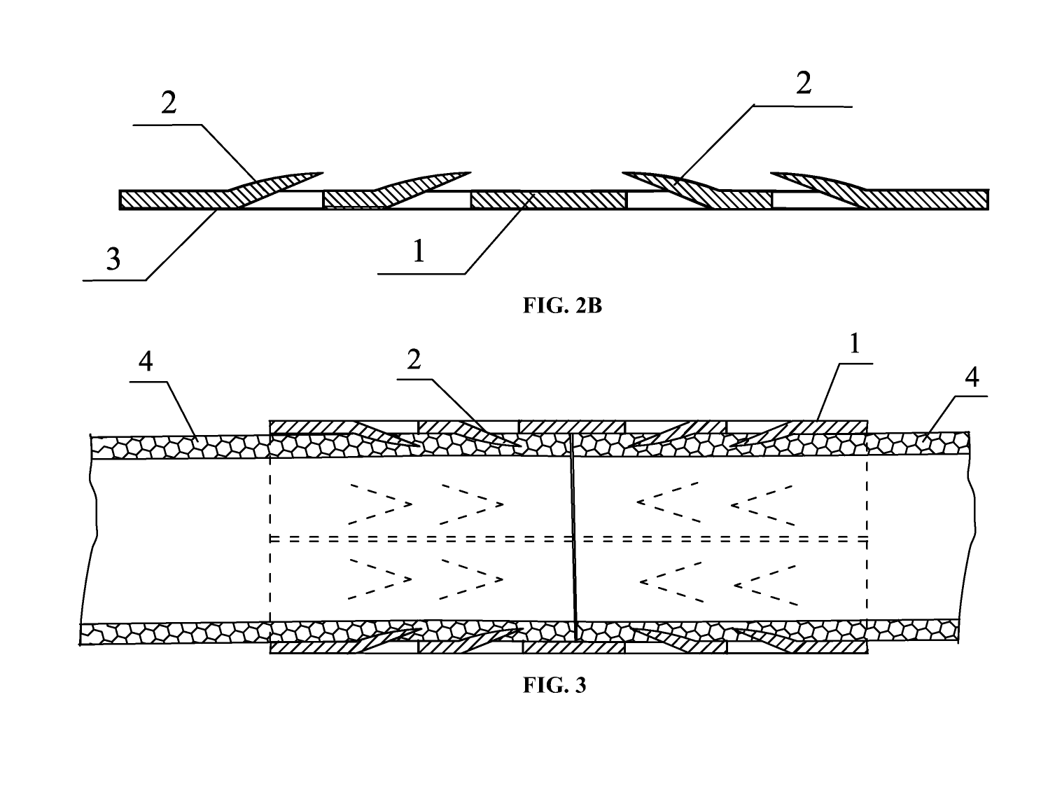

As shown in FIGS. 2A and 2B, a degradable surgical staple made from magnesium alloy comprises a base plate 1 made from magnesium alloy. The surface of the base plate 1 is provided with a plurality of opposite-distributed and staggered barbs 2. The base plate 1 and surfaces of the barbs 2 are coated with a drug layer 3. The drug layer can be absorbed by the body. The base plate 1 having the barbs 2 on the surface operates to roll inward or outward to form a tube for the stapling.

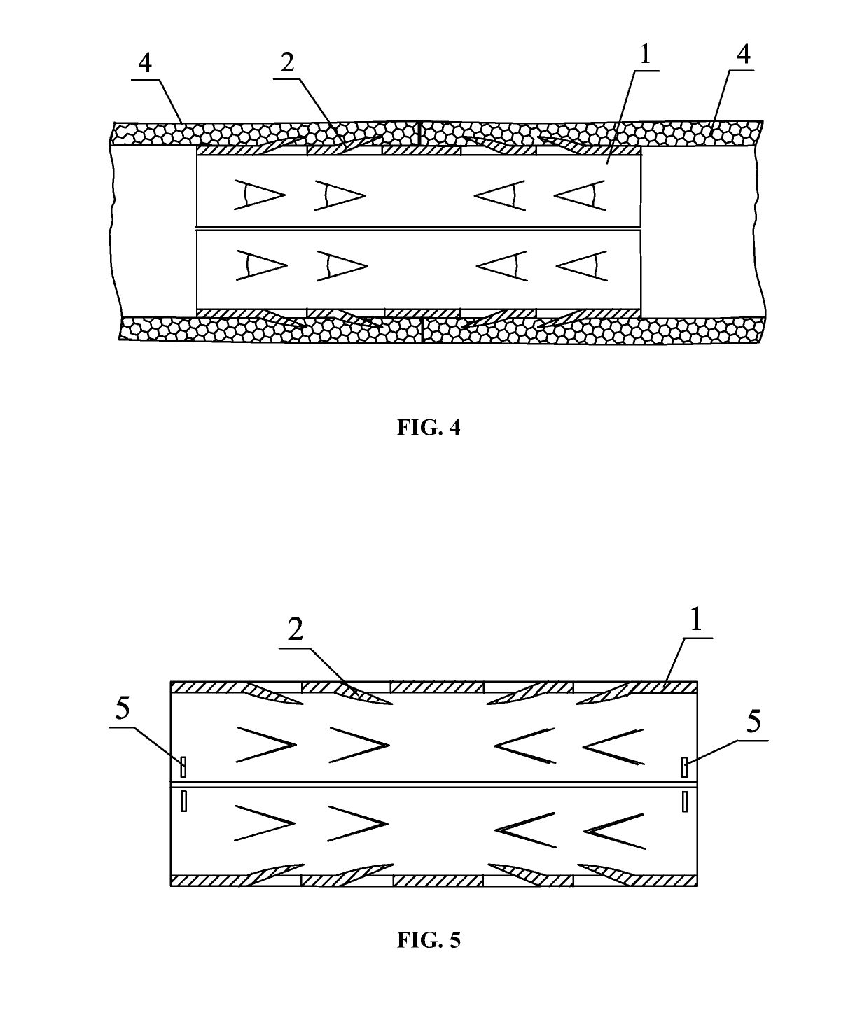

In operation, the base plate 1 of the staple is cut according to the size of the gastrointestinal organs. As shown in FIGS. 3-4, the base plate 1 is rolled inward to form a tube, and the barbs 2 protrude inward from an inner surface of the tube, then two ends of the gastrointestinal organ 4 are sheathed in the tube, slightly pulled, and thus hooked by the staggered barbs 2; or the base plate 1 is rolled outward to form a tube, and the barbs 2 protrude outward from an outer surface of the tube, then two ends of the gastrointestinal organ 4 are sleeved on the tube, slightly pulled, and thus hooked by the staggered barbs 2, thereby facilitating the stapling of gastrointestinal organ. The base plate in a tube shape is degraded or absorbed by the human body as the wound gradually heals up.

As shown in FIG. 5, the stapling of gastrointestinal organ having larger diameters uses the same method, the base plate 1 is also rolled up to form a tube, except that two ends of the tube are fixed by stapling nails 5.

The base plate 1 and surfaces of the barbs 2 in the invention are coated with the drug layer 3 which can be absorbed by the body. The drug layer can be replaced by the coating made from biocompatible and corrosion-resistant material which facilitates wound healing, or the coating made from anti-clotting, bactericidal, and bacteriostatic material. The drug layer and the coating bring no harm to the human body, instead, the drug layer and the coating with strong anti-clotting property and bactericidal capacity can protect the gastrointestinal organs from infections, thereby shortening the healing time of the wound.

Shaping a plurality of staggered barbs 2 on the base plate 1 is relatively easy. The base plate is rolled up to form a tube so that the staple is applicable for the stapling of gastrointestinal organ, as well as the stapling of esophagus, trachea, artery and vein, achieving a favorable medical stapling as well.

Unless otherwise indicated, the numerical ranges involved in the invention include the end values. While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A surgical staple, comprising a base plate made from magnesium alloy; wherein the base plate comprises a surface on which a plurality of opposite-distributed and staggered barbs is disposed.

2. The staple of claim 1, wherein the base plate and surfaces of the barbs are coated with a drug layer; the drug layer is absorbable by human body.

3. The staple of claim 1, wherein the base plate and surfaces of the barbs are coated with a drug layer which is absorbable by human body, or with a coating made from biocompatible and corrosion-resistant material, or with a coating made from anti-clotting, bactericidal, and bacteriostatic material.

4. The staple of claim 1, wherein the base plate having the barbs on the surface operates to roll inward or outward to form a tube for stapling of gastrointestinal organs.
